(19) United States
(12) Patent Application Publication
(10) Pub. No.: US 2011/0196032 A1
(43) Pub. Date: Aug. 11, 2011

(54) Pharmaceutical Dosage Form of an Antidepressant

(76) Inventors: Ashish Gogia, Andhra Prasesh (IN); Krishna Murthy Vanasi, Andhra Prasesh (IN); Sivakumaran Meenakshisunderam, Andhra Prasesh (IN)

(21) Appl. No.: 11/920,798

(22) PCT Filed: May 18, 2006
(86) PCT No.: PCT/IB2006/001527

§ 371 Date: Feb. 19, 2011

(30) Foreign Application Priority Data
May 20, 2005 (IN) 610/CHE/2005

(51) Int. Cl. A61K 31/343; C07D 307/06; B32B 5/00; A61P 25/24

(52) U.S. Cl. 514/469; 549/467; 428/402

(57) ABSTRACT

The present invention relates to pharmaceutical dosage forms of an antidepressant. More particularly, the present invention relates to pharmaceutical dosage forms of Escitalopram oxalate. The present invention also relates to a process for the preparation of pharmaceutical dosage forms of Escitalopram oxalate.

### FIELD OF THE INVENTION

The present invention relates to pharmaceutical dosage forms of an antidepressant. More particularly, the present invention relates to pharmaceutical dosage forms of Escitalopram oxalate.

The present invention also relates to a process for the preparation of pharmaceutical dosage forms of Escitalopram oxalate.

### BACKGROUND OF THE INVENTION

Citalopram is a well-known antidepressant drug that has the following structure: It is a selective, centrally active serotonin reuptake inhibitor.

Citalopram was first disclosed in U.S. Pat. No. 4,136, 193. This patent also describes the manufacture of tablets containing salts of citalopram.

Citalopram has been approved by USFDA in 1998. Subsequent to this, the S-enantiomer of citalopram, escitalopram was shown to have better activity profile and also been approved by FDA in 2002.

Escitalopram is chemically known as (+)-1-(3-Dimethylaminopropyl)-1-(4′-fluorophenyl)-1,3-dihydroisobenzofuran-5-carbonitrile. Escitalopram and its pharmaceutically acceptable salts are disclosed in U.S. Pat. No. 4,943,590, reissued as RE 34,712. This patent also describes the manufacture of tablets containing salts of Escitalopram. Escitalopram is an orally administered selective serotonin reuptake inhibitor (SSRI), and is indicated for the treatment of depression. Escitalopram is marketed as oxalate salt under the trade name LEXAPRO™.

Escitalopram is a poorly soluble drug and hence posing serious dissolution problems, which may affect bioavailability. For many drugs of low solubility, there is considerable evidence that the dissolution partially or completely controls the rate of absorption. Bioavailability can also be affected by a number of factors such as the amounts and types of adjuvants used, the granulation process, compression forces (in tablet manufacturing), the surface area available for dissolution.

Few approaches have been disclosed in the prior art addressing solutions to the solubility problems of Escitalopram. One such approach disclosed in U.S. Pat. No. 6,916,941 is using particle size of at least 40 μm and preparing the tablets by direct compression method. It further disclosed that escitalopram has significantly different solubility and salt formation properties from the citalopram racemate. For example, the only pharmaceutically crystalline salt known so far is the oxalate, whereas the citalopram racemate forms crystalline hydrobromide and hydrochloride salts as well.

The escitalopram oxalate product prepared by crystallization from acetone as outlined in U.S. Pat. No. 4,943,590 has, a very small particle, size around 2-20 microns resulting in poor flow properties. It is well recognized that preparation of tablets with a reproducible composition requires that all the dry ingredients have good flow properties. In cases, where the active ingredient has good flow properties, tablets can be prepared by direct compression of the ingredients. However, in many cases the particle size of the active substance is small, the active substance is cohesive or has poor flow properties.

None of the prior art references teaches the preparation of escitalopram oxalate by granulation technique to improve the flow properties.

### OBJECTIVE OF THE INVENTION

Accordingly, the main objective of present invention is to provide pharmaceutical dosage forms of escitalopram, which comply with the reference product in terms of in vivo parameters like bioequivalence and in vitro parameters like dissolution, disintegration.

Yet another objective of the present invention is to provide simple and efficient process for preparing pharmaceutical dosage forms of escitalopram, on a commercial scale.

### SUMMARY OF THE INVENTION

According to the main embodiment of the present invention, there is provided pharmaceutical dosage forms comprising escitalopram prepared by a granulation technique.

### DETAILED DESCRIPTION OF THE INVENTION

The particle size of the escitalopram oxalate used in the present invention is less than 20 μm.

In an embodiment of the present invention, there is provided pharmaceutical dosage forms of the Escitalopram oxalate, wherein the particle size distribution is such that at least 50% (median particle) of the particles are less than 15 microns and 95% of the particles are less than 20 microns.

In another embodiment of the present ignition, there is provided a pharmaceutical dosage forms comprising Escitalopram having uniform particle size distribution.

The dosage forms obtained by using the particle size distribution of Escitalopram according to the present invention result in homogeneous distribution of the drug substance in the tablet blend and produces tablets with uniform drug content.

Active substances with a small particle size mixed with excipients having a larger particle size will typically segregate or de-mix during the tabletting process. The problem of small particle size and poor flowability is conventionally solved by enlarging the particle size of the active substance, usually by granulation of the active ingredient either alone or in combination with a filler and/or other conventional tablet ingredients.

In an embodiment of the present invention, the granulation technique includes wet granulation or dry granulation process.

In an embodiment of the present invention, the pharmaceutical dosage form may further comprise one or more pharmaceutically acceptable excipients.

The pharmaceutically acceptable inert excipients may be one or more of binders, diluents, surfactants, lubricants/glidants and the like.

In yet another of the present invention, the pharmaceutical dosage form of escitalopram further contains a wetting agent. The use of a wetting agent reduces the surface tension of water and therefore increases adhesion of water to the escitalopram surface. The use of a wetting agent may also be useful in improving the bioavailability of escitalopram.

The diluents used according to the present invention are selected from calcium phosphate-dibasic, calcium carbonate, lactose, sucrose, cellulose-microcrystalline, cellulose powdered, silicified microcrystalline cellulose, calcium silicate, kaolin, starch, starch pregelatinized, polyols such as mannitol, sorbitol, xylitol, maltitol, sucrose and combinations thereof.

Suitable disintegrants used in accordance with the present invention are selected from croscarmellose sodium, crospovidone, sodium starch glycolate, sodium carboxymethylcellulose, hydroxypropylcellulose, xanthan gum, alginic acid, alginates, carbopols and the like or combination thereof.

Suitable binders according to the present invention are selected from methyl cellulose, hydroxypropyl cellulose, hydroxypropyl methyl cellulose, polyvinylpyrrolidone, gelatin, gum arabic, ethyl cellulose, polyvinyl alcohol, starch, pregelatinized starch, agar, tragacanth, sodium alginate, propylene glycol, alginate, plasdone and the like.

Suitable lubricants according to the present invention are selected from sodium lauryl sulfate, talc, magnesium stearate, sodium stearyl fumarate, stearic acid, glyceryl behenate, hydrogenated vegetable oil, zinc stearate and suitable glidants include colloidal silicon dioxide and talc.

Suitable wetting agents of the present invention are selected from anionic, cationic or non-ionic surface-active agents or surfactants. Suitable anionic surfactants include sodium lauryl sulfate, sodium laurate, sodium stearate, potassium stearate, sodium oleate and the like. Suitable cationic surfactants include benzalkonium chloride, bis-2-hydroxyethyl oleyl amine and the like. Suitable non-ionic surfactants include polyoxyethylene sorbitan fatty acid esters, fatty alcohols such as lauryl, cetyl and stearyl alcohols; glyceryl esters such as the naturally occurring mono-, di-, and tri-glycerides; fatty acid esters of fatty alcohols and other alcohols such as propylene glycol, polyethylene glycol, sorbitan, sucrose, and cholesterol.

In yet another embodiment of the present invention, the dry granulation technique comprises slugging and compaction. The compaction process of the present comprises the steps of blending escitalopram oxalate along with or without excipients, compacting the blend and sieving the granules to obtain uniform particle size, blending the granules with extragranular excipients and compressing the blend into tablets.

In yet another embodiment of the present invention, there is also provided a method for treating patients suffering from depression comprising administering a dosage form of escitalopram oxalate of the present invention.

In yet another embodiment of the present invention, there is also provided a process for the preparation of pharmaceutical dosage form comprising escitalopram by wet granulation techniques, which comprises the steps of:
i) mixing escitalopram oxalate with the one or more excipients
ii) granulating the blend obtained in step (i) using solvent and optionally a binder,
iii) drying the granules obtained in step (ii),
iv) mixing .the granules of step (iii) with one or more pharmaceutically acceptable excipients, .
v) lubricating the blend of step (iv) and
vi) compressed the blend of step (v) into tablets.

In an embodiment of the present invention, the solvents used for granulation may be selected from water or organic solvents such as acetone, alcohol, isopropyl alcohol and the mixture thereof.

In yet another embodiment of the present invention, the tablets include uncoated tablets, film coated tablets coated with polymers selected from hydroxypropyl methyl cellulose, hydroxypropyl cellulose, polyvinylpyrrolidone, polyvinyl alcohol, ethyl cellulose, polyethylene oxide and the like.

### Example 1

### Formulation of Film Coated Tablets of Escitalopram Oxalate by Compaction

| Ingredients | Quantity (mg/tablet) |
|---|---|
| Escitalopram | 20.0 |
| Microcrystalline cellulose | 30.0 |
| Lactose anhydrous | 35.0 |
| Crospovidone | 3.0 |
| Colloidal silicon dioxide | 2.0 |
| Magnesium stearate | 4.00 |

The processing steps that are involved in making film coated tablets of escitalopram oxalate disclosed above are given below:
(i) escitalopram oxalate, microcrystalline cellulose, lactose, colloidal silicon dioxide, half the quantity of the crospovidone were blended,
(ii) the blend obtained in step (i) was compacted and sieved to obtain uniform particle size through a suitable mesh,
(iii) the granules of step (ii) were blended with microcrystalline cellulose and remaining quantity of crospovidone,
(iv) lubricated blend of the step (iii) with magnesium stearate,
(v) compressed the blend of step (iii) into tablets and
(vi) the compressed tablets are further film coated.
   The formulations described in examples 2 to 6 were prepared using the procedure similar to the one described in example 1.

### Example 2

| S. No. | Ingredients | Quantity (mg) |
|---|---|---|
| | Escitalopram oxalate | 25.54 |
| | Silicified microcrystalline cellulose | 99.5 |
| | Croscarmellose sodium | 9.0 |
| | Purified water | Qs |
| | Extragranular | |
| | Silicified microcrystalline cellulose | 99.5 |
| | Microcrystalline cellulose | 0.0 |
| | Colloidal silicon dioxide | 0.0 |
| | Talc | 14.0 |
| | Magnesium stearate | 2.5 |
| | Core tablet wt(mg) | 250.0 |

### Example 3

| S. No. | Ingredients | Quantity (mg) |
|---|---|---|
| | Escitalopram oxalate | 25.54 |
| | Silicified microcrystalline cellulose | 99.5 |
| | Croscarmellose sodium | 10.0 |
| | Purified water | qs |
| | Extragranular | |
| | Silicified microcrystalline cellulose | 0.0 |
| | Microcrystalline cellulose | 96.21 |
| | Colloidal silicon dioxide | 1.25 |
| | Talc | 5.0 |
| | Magnesium stearate | 2.5 |
| | Core tablet wt(mg) | 240.0 |

### Example 4

| S. No. | Ingredients | Quantity (mg) |
|---|---|---|
| | Escitalopram oxalate | 25.54 |
| | Silicified microcrystalline cellulose | 99.5 |
| | Croscarmellose sodium | 10.0 |
| | Purified water | Qs. |
| | Extragranular | |
| | silicified microcrystalline | 0.0 |
| | Microcrystalline cellulose cellulose | 107.46 |
| | Colloidal silicon dioxide | 1.25 |
| | Talc | 5.0 |
| | Magnesium stearate | 1.25 |
| | Core tablet wt(mg) | 250.0 |

### Example 5

| S. No. | Ingredients | Quantity (mg) |
|---|---|---|
| | Escitalopram oxalate | 25.54 |
| | silicified microcrystalline cellulose | 99.5 |
| | Croscarmellose sodium | 10.0 |
| | Purified water | Qs |
| | Extragranular | |
| | Silicified microcrystalline cellulose | 0.0 |
| | Microcrystalline cellulose | 106.21 |
| | Colloidal silicon dioxide | 1.25 |
| | Lubricant | |
| | Talc | 5.0 |
| | Magnesium stearate | 2.5 |

### Example 6

| | | Qty per unit |
|---|---|---|
| S. No. | Ingredients | (mg) |
| 1. | Escitalopram oxalate | 25.54 |
| 2. | Silicified microcrystalline cellulose | 99.5 |
| 3. | Croscarmellose sodium | 10.0 |
| 4. | Purified water | q.s |
| | Extragranular | |
| 5. | Microcrystalline Cellulose | 106.21 |
| 6. | Colloidal silicon dioxide | 1.25 |
| 7. | Talc | 5.0 |
| 8. | Magnesium stearate | 2.5 |
| | Core. Tablet weight | 250.0 |

### Dissolution Profile of Escitalopram Oxalate Tablets

The tablets were subjected to an in vitro dissolution method to determine the rate at which the Escitalopram oxalate was released from the tablets. The tablets were placed into a dissolution medium of 900 ml 1 0.1 N HCL and stirred with paddles at 50 rpm (USP 2 apparatus). The dissolution profile is given in Table 1

TABLE 1
Escitalopram Oxalate Tablets 20 mg

| | % Release | | | | |
|---|---|---|---|---|---|
| | Time in minutes | | | | |
| Example | 5 | 10 | 20 | 30 | 40 |
| Example 2 | 93 | 95 | 99 | 99 | 100 |
| Example 3 | 89 | 94 | 97 | 99 | 100 |
| Example 4 | 88 | 94 | 97 | 99 | 100 |
| Example 5 | 93 | 98 | 102 | 103 | 104 |
| Lexapro ® | 87 | 94 | 97 | 98 | 99 |

1. A pharmaceutical dosage form comprising Escitalopram prepared by a granulation technique.

2. The dosage form as claimed in claim 1, wherein the article size of the Escitalopram oxalate used is less than 20 μm.

3. The dosage form as claimed in claim 1, wherein the granulation technique includes wet granulation or dry granulation process.

4. The dosage form as claimed in claim 1, wherein further comprise one or more pharmaceutically acceptable excipients selected from binders, diluents, surfactants, lubricants/glidants.

5. The dosage form as claimed in claim 1, wherein the diluents used is selected from the group consisting of calcium phosphate-dibasic, calcium carbonate, lactose, sucrose, cellulose-microcrystalline, cellulose powdered, silicified microcrystalline cellulose, calcium silicate, kaolin, starch, starch pregelatinized, mannitol, sorbitol, xylitol, maltitol, sucrose or combination thereof.

6. The dosage form as claimed in claim 1, wherein the disintegrants used is selected from the group consisting of croscarmellose sodium, crospovidone, sodium starch glycolate, sodium carboxymethylcellulose, hydroxypropylcellulose, xanthan gum, alginic acid, alginates, carbopols or combination thereof.

7. The dosage form as claimed in claim 1, wherein the binder used is selected from the group consisting of methyl cellulose, hydroxypropyl cellulose, hydroxypropyl methyl cellulose, polyvinylpyrrolidone, gelatin, gum arabic, ethyl cellulose, polyvinyl alcohol, starch, pregelatinized starch, agar, tragacanth, sodium alginate, propylene glycol, alginate, plasdone.

8. The dosage form as claimed in claim 1, wherein the lubricant used is selected from the group consisting of sodium lauryl sulfate, talc, magnesium stearate, sodium stearyl fumarate, stearic acid, glyceryl behenate, hydrogenated vegetable oil, zinc stearate.

9. The dosage form as claimed in claim 1, further comprise surfactant.

10. The dosage form as claimed in claim 9, wherein the surfactant is selected from the group consisting of sodium lauryl sulfate, sodium laurate, sodium stearate, potassium stearate, sodium oleate benzalkonium chloride.

11. A process for the preparation of pharmaceutical dosage form comprising Escitalopram by wet granulation techniques, which comprises the steps of:
i) mixing Escitalopram oxalate with the one or more excipients
ii) granulating the blend obtained in step (i) using solvent and optionally a binder,
iii) drying the granules obtained in step (ii),
iv) mixing the granules of step (iii) with one or more pharmaceutically acceptable excipients,
v) lubricating the blend of step (iv) and
vi) compressed the blend of step (v) into tablets.
